(19) United States
(12) Patent Application Publication
(10) Pub. No.: US 2012/0089005 A1
(43) Pub. Date: Apr. 12, 2012

(54) Multi-Modal Medical Imaging and Type Detection

(75) Inventors: Rahim Bagheri, Carlsbad, CA (US); Ghobad Heidari-Bateni, San Diego, CA (US)

(73) Assignee: WiLinx Corporation, Carlsbad, CA (US)

(21) Appl. No.: 13/269,842

(22) Filed: Oct. 10, 2011

Related U.S. Application Data
(60) Provisional application No. 61/391,921, filed on Oct. 11, 2010.

(51) Int. Cl. A61B 6/00

(52) U.S. Cl. 600/407

(57) ABSTRACT

A multi-modal medical imaging and type detection have been described have been disclosed. By combining a broadband radio imaging system and one or more other modalities of image processing an enhanced image and/or tissue classification can be produced.

### RELATED APPLICATION

The present application for patent is related to U.S. Patent Application No. 61/391,921 titled “Multi-Modal Medical Imaging and Type Detection” filed Oct. 11, 2010, pending, by the same inventors and which is hereby incorporated herein by reference.

### FIELD OF THE INVENTION

The present invention pertains to medical imaging. More particularly, the present invention relates to Multi-Modal Medical Imaging and Type Detection.

### BACKGROUND OF THE INVENTION

Currently, there are several modes of imaging the internal tissues/organs of the body: Xray/CT scan, MRI, PET scan, and ultrasonography.

Currently the only way to detect the type of a tissue is by visual inspection on a high-resolution image (from, say, ultrasound, Xray, CT, MRI, or PET) and then doing a biopsy to confirm determination. Visual inspection of an image is prone to errors stemming from human judgment or potential artifacts of the image.

This presents a technical problem needing a technical solution.

### BRIEF DESCRIPTION OF THE DRAWINGS

The invention is illustrated by way of example and not limitation in the figures of the accompanying drawings in which:

FIG. 1 illustrates a network environment in which the method and apparatus of the invention may be implemented.

FIG. 2 is a block diagram of a computer system in which some embodiments of the invention may be implemented.

FIG. 3 is a Broadband Radio Imaging (BRI) overview.

FIG. 4, FIG. 5, FIG. 6, FIG. 7, FIG. 8, FIG. 9, and FIG. 10 illustrate various embodiments of the invention.

FIG. 11 illustrates various embodiments of the invention.

### DETAILED DESCRIPTION

A multi-modal medical imaging and type detection is disclosed.

A modality is used that uses a completely different method of medical imaging—radio waves. In particular Broadband Radio Imaging (BRI) uses very broadband radio waves to image body tissues/organs.

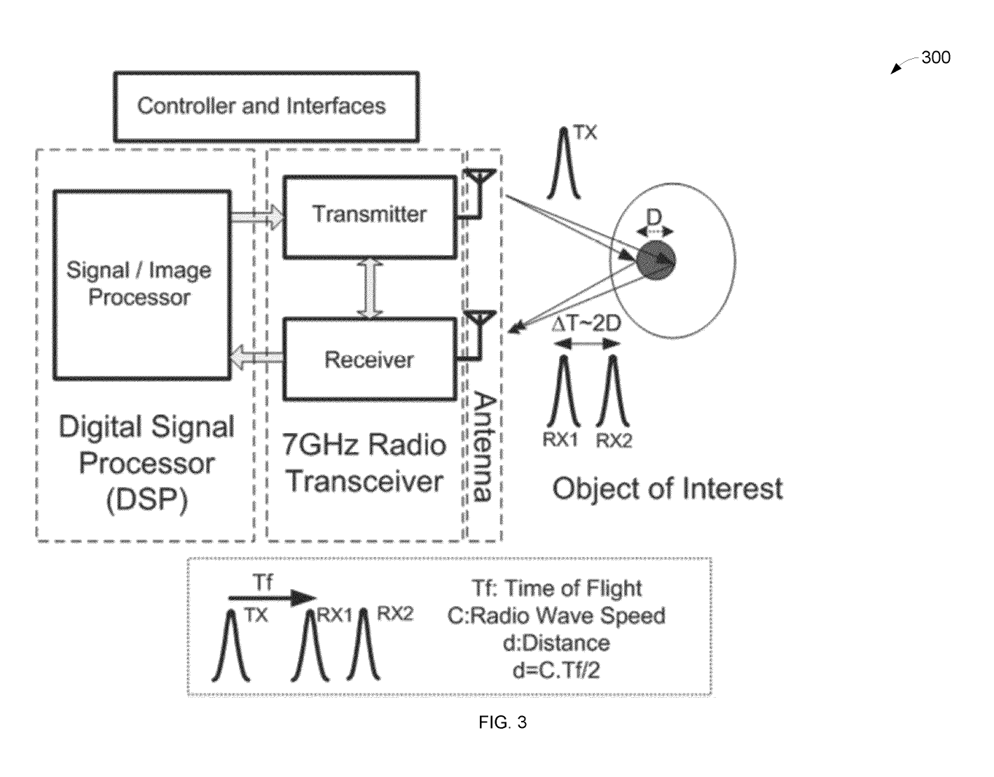

BRI can perform medical imaging in a number of technologies. The first and more straightforward technology is to measure the time of flight of signals as shown in FIG. 3 generally at 300. In FIG. 3, the BRI system components are detailed: Transmitter, Receiver, Signal/image Processor, and Controller and Interfaces. Due to the large bandwidth of the signal transmitted, the time resolution of the pulses received is extremely high. Plus, since the signal is traveling inside the body (e.g., human, or animal), the speed of the signal is reduced, making the resolution even higher.

The other technology to implement in BRI (either as a replacement or augment to time of flight) is to solve the inverse scattering problem of determining the dielectric constants from the knowledge of the electromagnetic fields around the object of interest. Thus in one embodiment of the invention, by measuring the EM (electromagnetic) field all around the object, and taking advantage of any a priori knowledge that might be available about the interior of the object, the dielectric constants of the different parts of the object may be determined. This determination can then be mapped to the tissue classification, for example, but not limited to, cancerous versus normal, etc.

This leads to one advantage of BRI the capability to distinguish different types of tissues from each other. This is because radio waves are sensitive to the dielectric constants of different tissues, and these dielectric constants vary drastically within the body. For example, the dielectric constant of a cancer tissue may be 5 times higher than that of a normal tissue.

In this invention, we use the combination of BRI and another imaging technique (e.g., ultrasound, Xray, CT, MRI, PET) to get a better image (image enhancement) or to detect the type of a body tissue (tissue classification). At least two beneficial methods of combining imaging modalities can be conceived as such. These are described below (tissue classification, and image enhancement). Within each technique, multiple alternatives may exist that also are exemplified below. However the invention is not so limited and other combinations are possible.

It is understood that the examples given below are for clarification purposes and other similar combinations and applications can be envisaged by someone who is familiar with the art. In particular, although examples are about human/animal body imaging, nothing precludes us from using the techniques described here to apply them to inanimate objects and/or plants, etc.

Tissue Classification

In BRI-based image reconstruction of an object, one needs to estimate both the location and the dielectric constant of the components of that objects since both are unknown prior to the reconstruction of the image. As a result it requires extensively computational algorithms to reconstruct the image. In return, the image carries both the image shape/location (geometrical information of the components) of the object, as well as the type of component (based on its dielectric constant).

On the other hand, other imaging modalities such as ultrasound or X-ray may provide a high resolution image (only geometrical information) with lower computation complexity but cannot provide much information about the type of the tissues involved. With the combined use of BRI and one or more other modalities we can provide a high resolution image as well as the tissue type at reduced computation complexity. That is, the use of two or more modalities of imaging can reduce the overall complexity of the imaging and type detection tasks.

In another embodiment, ultrasound can be used to give the boundaries of the tissue (e.g., tumor) of interest, and BRI is then enabled to classify the tissue type (using its dielectric constant). For example, ultrasound may provide the image shape (say location and shape of various organs/tissues inside the body). An algorithm will then use the geometrical information received from the ultrasound image and the raw measurement results from BRI to detect the tissue type. To be more specific, since the detailed shape of the object's interior (including tumors, blood clots, etc) is known from ultrasound measurement at a high resolution, BRI data will only be used to estimate the dielectric constants (one set of unknowns) with much less computational effort (compared to using BRI alone).

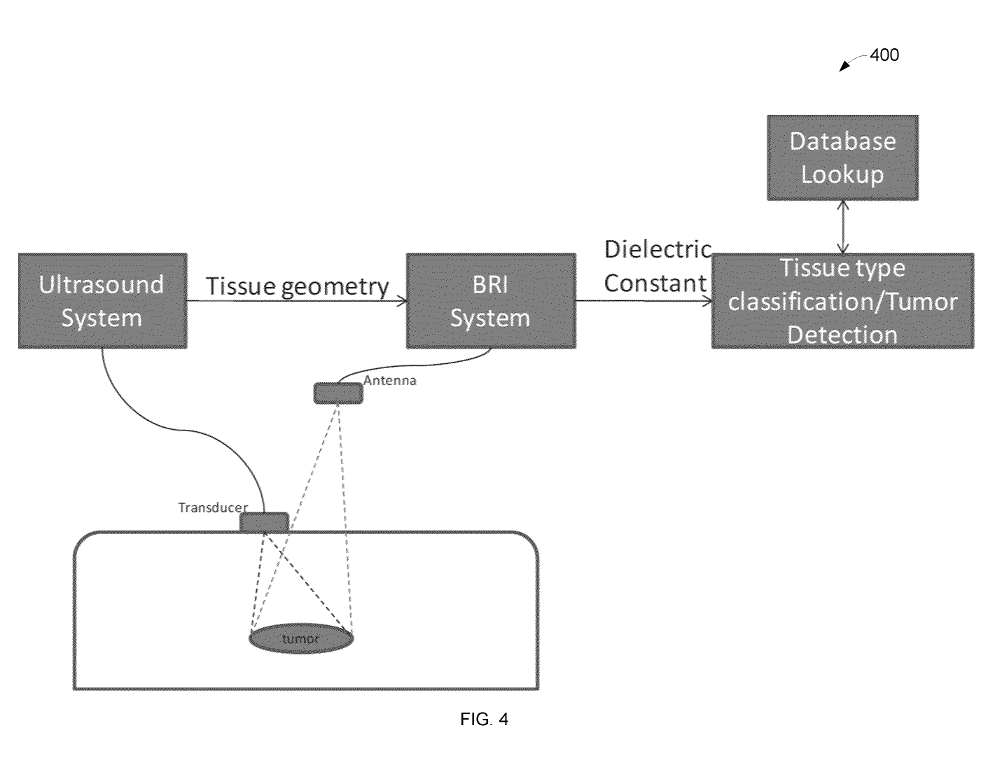

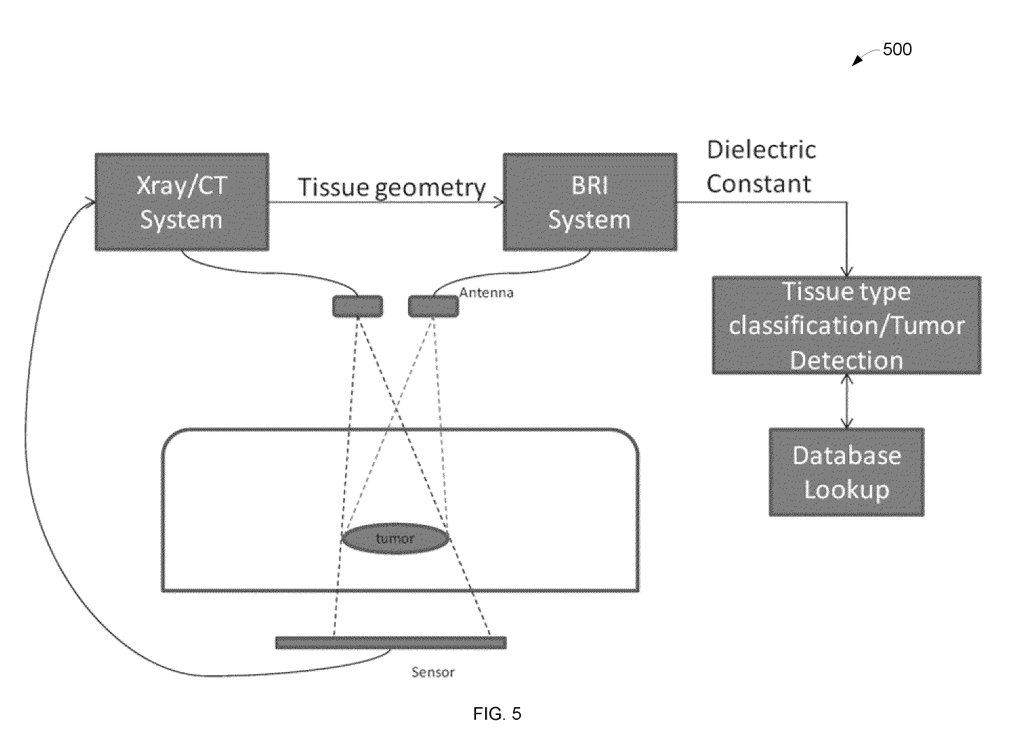

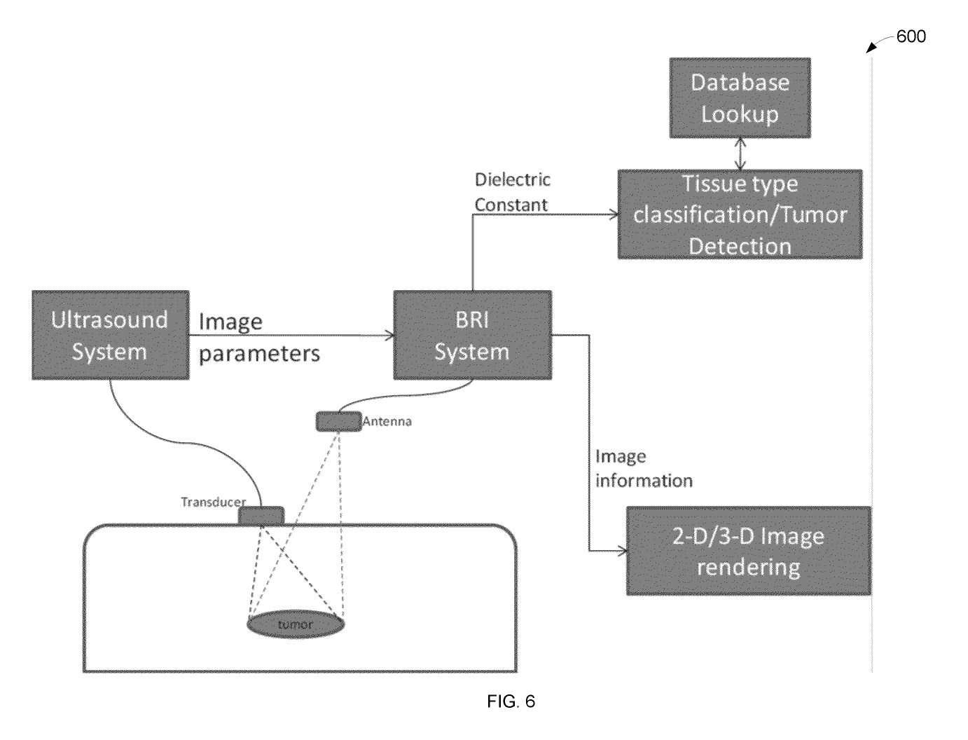

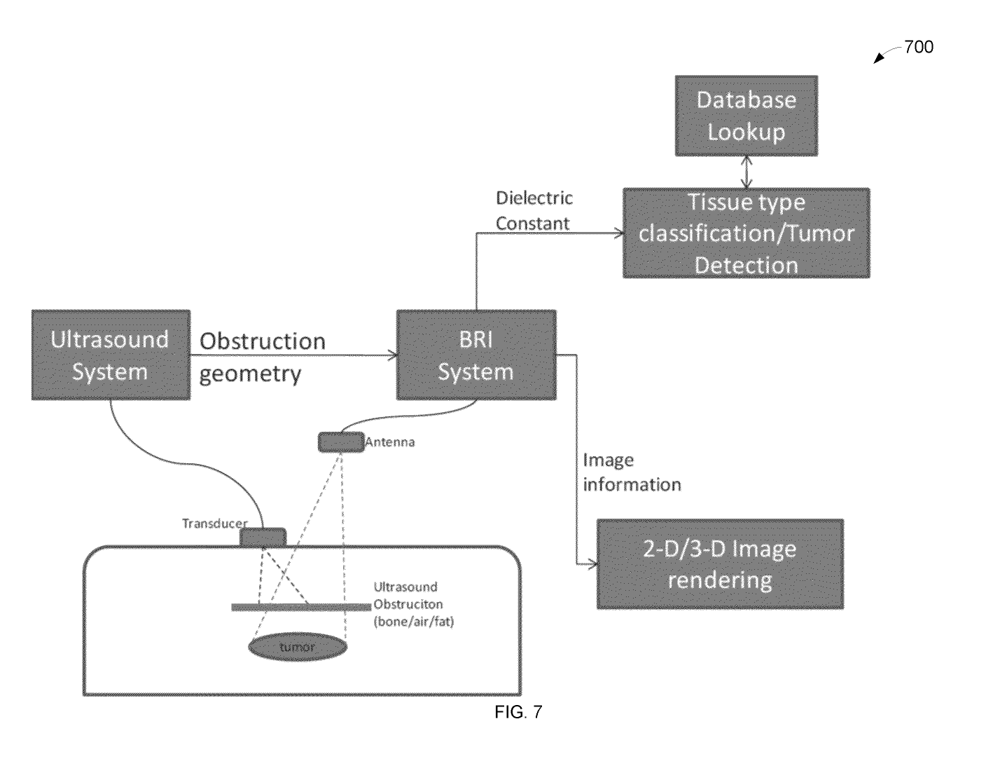

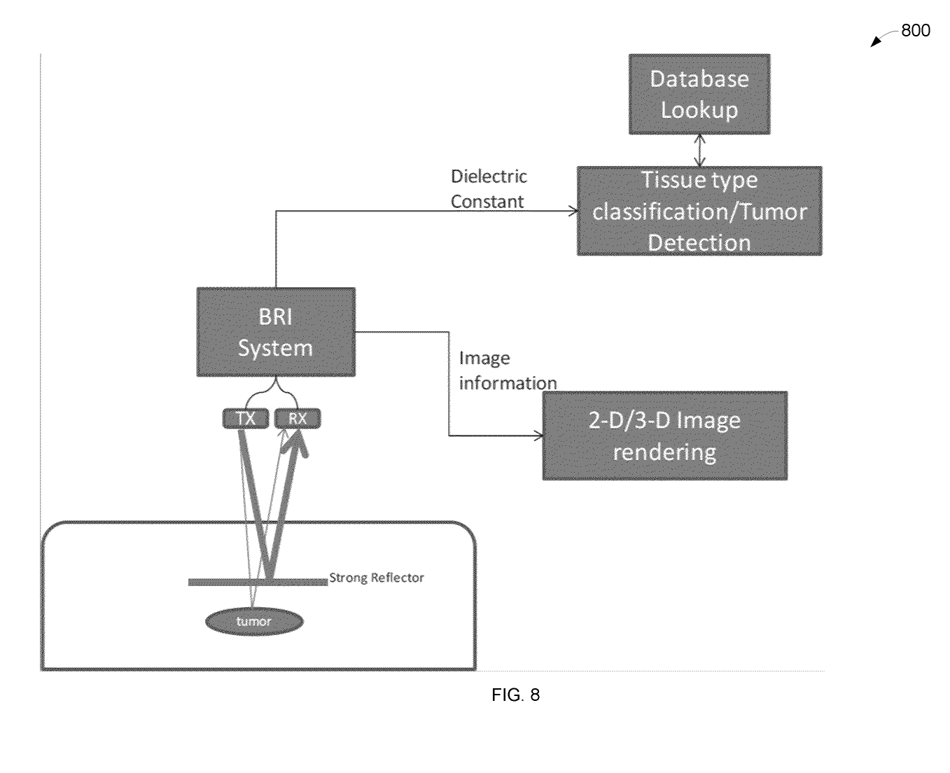

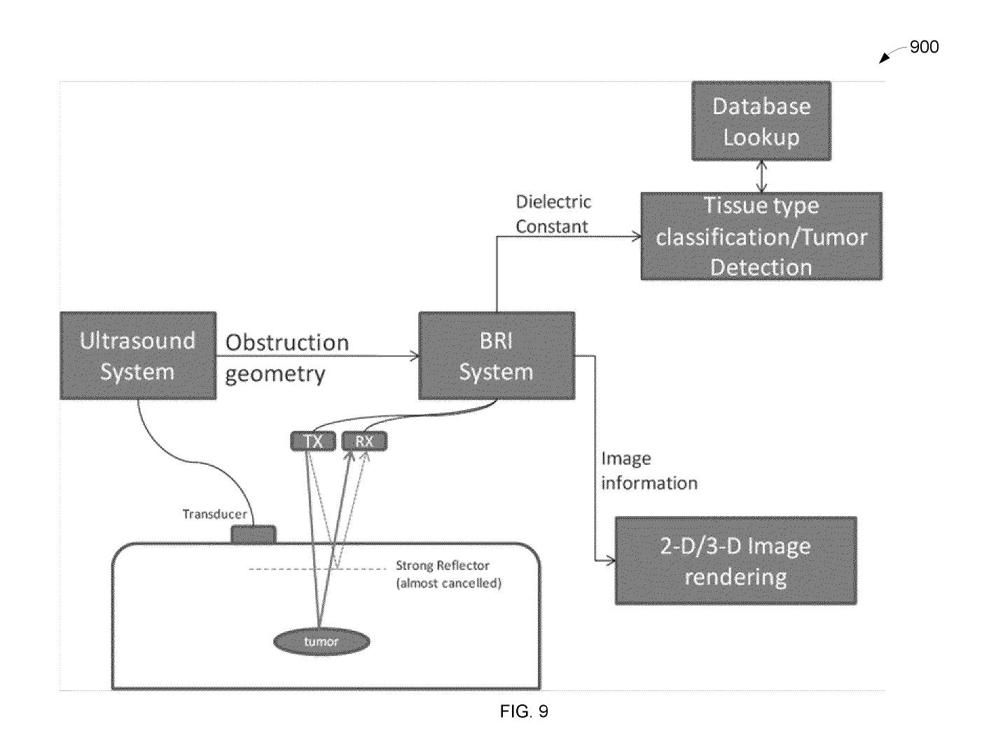

FIG. 4 depicts, generally at 400, an example of such an embodiment in which a tumor within an organ or body is being detected by the combination of an ultrasound system (US) and a BRI system. The US detects the geometry of the tumor within the organ/body. This includes the location and the boundaries of the tumor. This information is then passed on to the BRI system, which uses this information to detect the dielectric constant of the tissue of interest (the tumor). This value is then looked up in a database that, among other things, uses the dielectric constant value and all the pertinent information available about the tissue/object of interest to lookup the classification information of tissue of interest. Tissue Type Classification/Tumor Detection uses the information from the database to classify the tissue as, for example, cancerous or normal.

In another embodiment of the invention, use may be made of Xray, or MRI to image a breast and to feed the geometrical information to BRI to help with determination of the dielectric constants within the breast tissue. As such, a cancerous tissue, which has a dielectric constant much higher than that of normal tissue, may be detected using BRI.

FIG. 5 shows, generally at 500, this setup with a combination of an Xray or CT system with BRI system, in which the Xray/CT system provides the geometric information to BRI system, and BRI system detects the dielectric constant of the tissue of interest. As before, the result is a classification of the tissue as either benign or malignant. Note that, unlike the ultrasound system, the use of the Xray/CT system allows the imaging apparatus to be away from the patient of object under examination.

Image Enhancement

BRI has certain advantages and disadvantages relative to other medical imaging modalities. For example, compared to ultrasonography, BRI can image in air, bone and fat, something that ultrasound is incapable of performing. On the other hand, BRI's images may not be as sharp as those of ultrasound.

We combine the imaging capabilities of BRI and one or more of the other imaging modalities to generate a composite image that is more resolved and/or more complete than otherwise.

For example, an ultrasound image or its parameters can be fed to a BRI imaging system to allow it to generate a more resolved and/or more complete image. The boundary information from an ultrasound image can help BRI with its own image resolution or help to reduce the complexity of its imaging algorithms.

FIG. 6 illustrates, generally at 600, this embodiment. In this case, the ultrasound system makes certain imaging analysis and provides certain image parameters to the BRI system. These parameters are then used by the BRI system to make its own image detection with far less complexity or far more accuracy. As a result, the image rendered from the BRI system is more quickly obtained and/or more highly resolved. The BRI system can still provide the additional information about the dielectric constant of the tissue of interest to allow tissue type classification.

As another example, one can use an ultrasound image and combine it with a BRI image to gain a much better image. Ultrasound may contain a noisy image in areas where bone/air/fat exists. BRI can fill in those areas with much better clarity and accuracy.

Consider the example of FIG. 7, generally at 700, which shows the tumor (or tissue/organ to be imaged and analyzed) is positioned behind an obstruction. This obstruction could be made of bone, fat, air or anything else which ultrasound does not penetrate. As such, the ultrasound system can only provide information (including geometry) about the obstruction object and all other tissue/organs above it. The BRI system on the other hand can penetrate the obstruction and reach the tissue/organ/tumor of interest. Using the geometrical information that the ultrasound system can provide the BRI system is then able to provide a more resolved image of the tissue/organ/tumor of interest with less implementational complexity and/or in a shorter period of time.

As another example of the use of the combination of two imaging modalities, consider FIG. 8, generally at 800, in which, a BRI system is using its pair of transmitter (TX) and receiver (RX) to detect, image, and identify/classify a tumor within the body. There is an obstruction in between the BRI TX and the tumor. As a result, there is a strong reflection from the obstruction back to the RX. This strong signal will dominate the frontend of the RX and will cause problems in the acquisition/detection of the weak signal from the tumor.

As such, the BRI system may have to resort to very complex and time-consuming iterative signal processing techniques to detect the strong reflector and cancel its interference from the received signal to be able to finally detect the signal from the tumor. It is conceivable that there may be scenarios in which the cancellation of the strong reflections may not be achievable to be able to capture the necessary signals from the tumor.

To alleviate these problems, a combination of imaging modalities may be used, as exemplified in FIG. 9, generally at 900. Here, an ultrasound imaging system is combined with a BRI system. The ultrasound system provides accurate geometric information about the obstruction layer(s) to the analog/digital acquisition module of the BRI system so as to allow the BRI system to remove/cancel the strong reflections it receives from those layers. This will reduce the overall complexity of the BRI system's signal acquisition/signal processing. The use of ultrasound in effect quickly provides the information that otherwise would take many iterations and much longer to achieve using BRI system alone, if at all.

In the case where the BRI system uses reverse scattering techniques to determine the dielectric constants and to create a 2-D/3-D image of the different layers and objects/tissues inside the body, the use of the multi-modal approach is also very useful. Consider FIG. 10, generally at 1000, in which, the tumor is inside layers of different types of tissues with different dielectric constants (ε1, ε2, ε3, ε4 in FIG. 10).

Thus, assuming that backscattering analysis is being applied to the received signals in the BRI system based on the iterative forward scattering analysis, an estimated model of the whole object including the ultrasound-generated precise boundary around the tumor can be fed to the BRI system to help it through its iterations. In other words, the signal processor of the BRI system would get the geometrical information of the areas imaged by the ultrasound system. The BRI system may either have the exact/estimated values of the corresponding dielectric constants (ε1, ε2, ε3 in FIG. 10) as a priori knowledge, or it may estimate them as part of its inverse scattering algorithm. Either way, the ultrasound input to the BRI system will significantly lower the analysis complexity since the number of unknowns for the BRI signal processing algorithm is reduced.

This invention disclosure need not be confined to medical imaging. Any industrial or other types of imaging of hidden components may also take advantage of this technique.

Ultrasound and BRI was just an example. In other embodiments, BRI may be combined with/assisted by other imaging modalities such as Xray, CT scan, MRI, PET, etc. Or, BRI may receive the required geometrical information using non-imaging approaches, such as precise manual measurement.

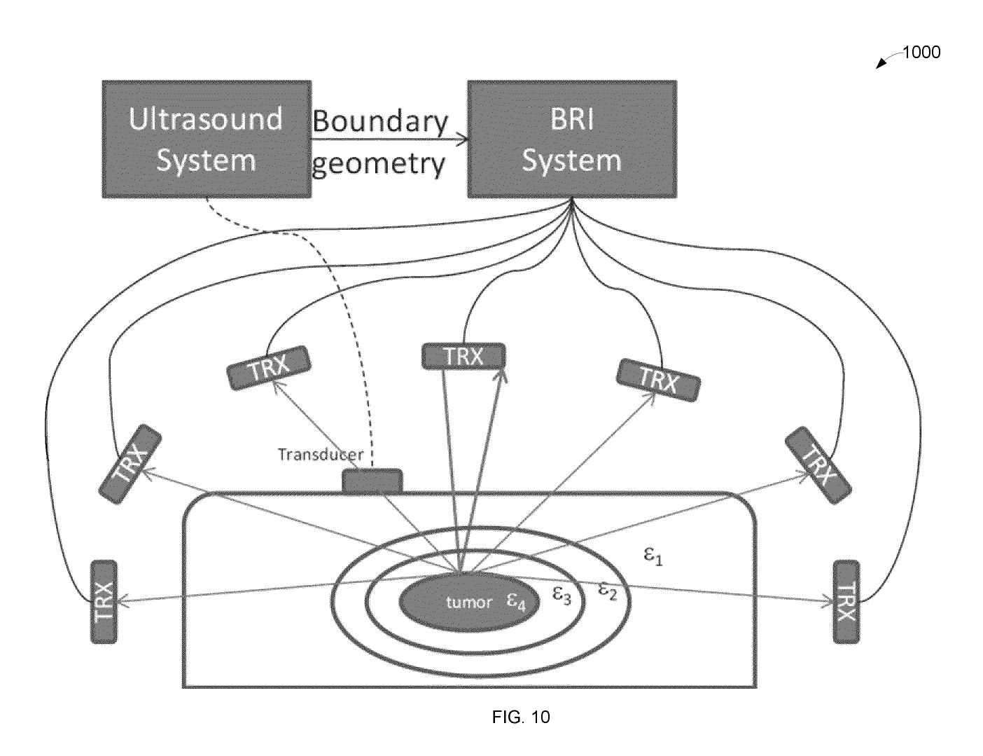

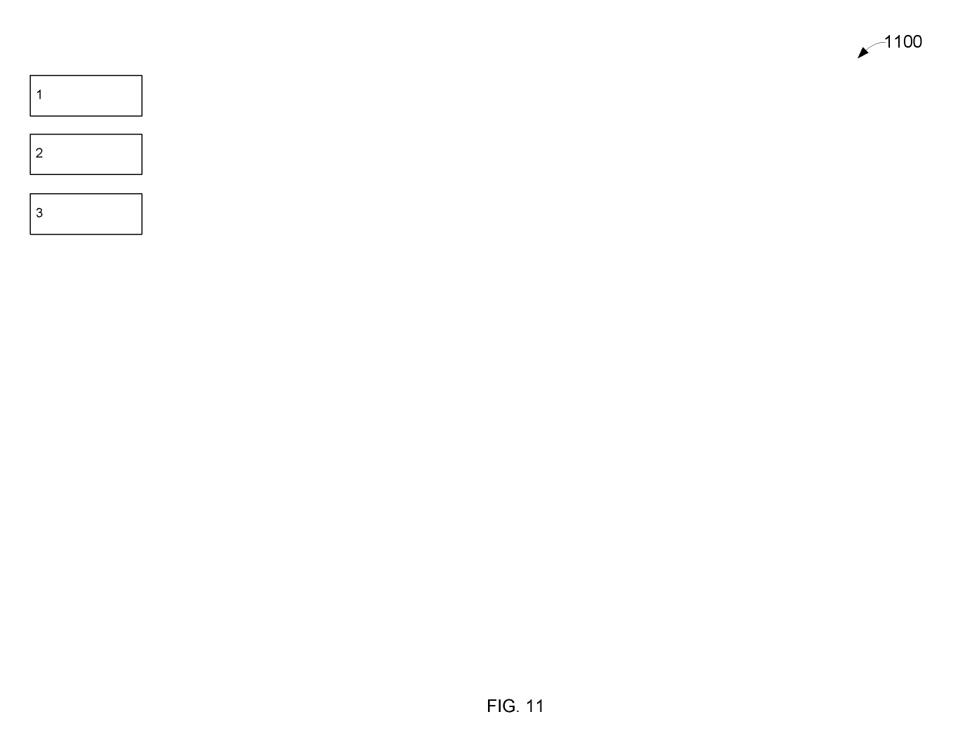

FIG. 11 illustrates various embodiments of the invention where:

1 denotes in one embodiment 1. A method for imaging comprising:

generating geometric information using a non-broadband radio imaging system;

sending said generated geometric information to a broadband radio imaging system;

using said broadband radio imaging system to generate dielectric constant information based on said generated geometric information.

2 denotes in one embodiment 2. An apparatus comprising:

a non-broadband radio imaging system having a geometry output; and

a broadband radio imaging system having a geometry input and a dielectric constant output, wherein said geometry input is coupled to said geometry output.

3 denotes in one embodiment 3. A method for combining a broadband radio imaging system and one or more other modalities of image processing to produce tissue classification.

Thus a Multi-Modal Medical Imaging and Type Detection have been described.

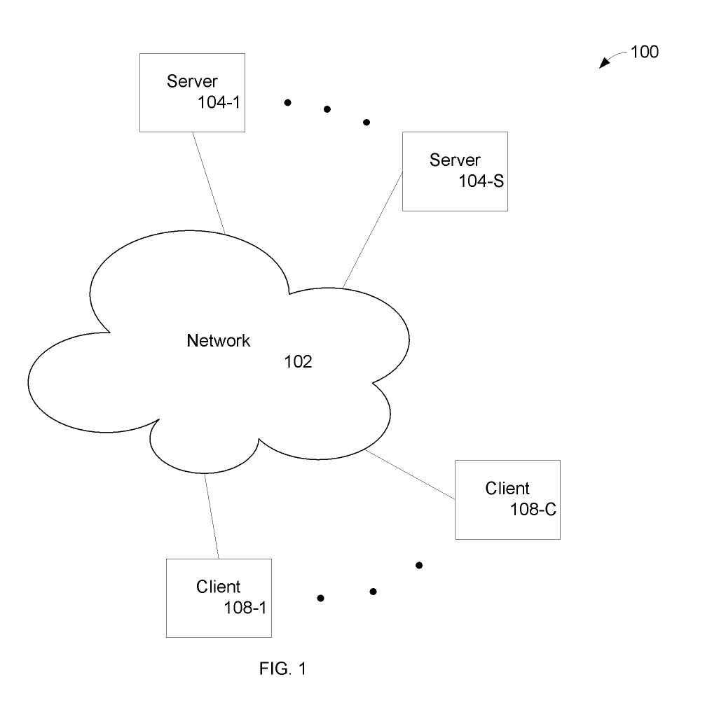

FIG. 1 illustrates a network environment 100 in which the techniques described may be applied. The network environment 100 has a network 102 that connects S servers 104-1 through 104-S, and C clients 108-1 through 108-C. More details are described below.

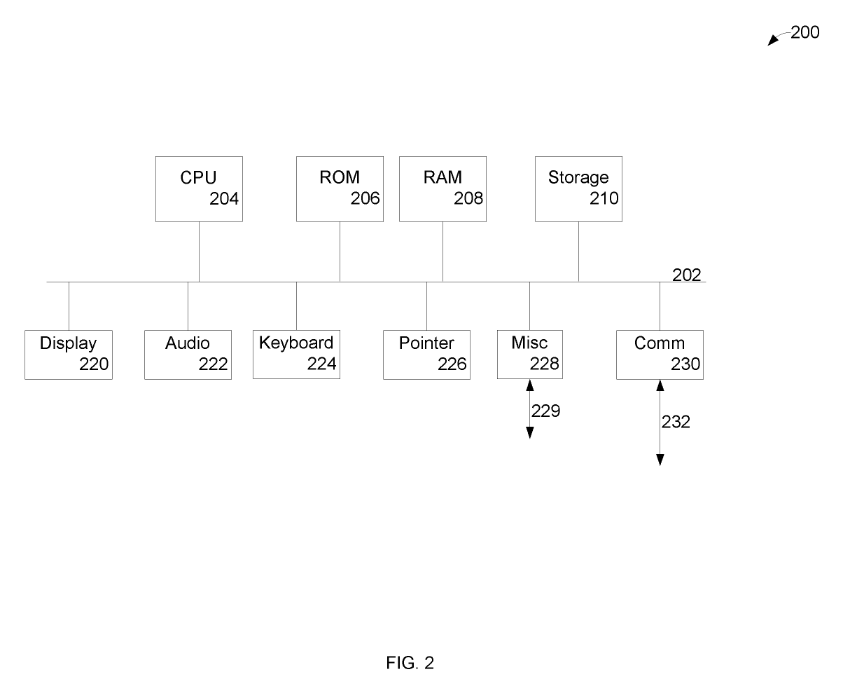

FIG. 2 is a block diagram of a computer system 200 in which some embodiments of the invention may be implemented and which may be representative of use in any of the clients and/or servers shown in FIG. 1, as well as, devices, clients, and servers in other Figures. More details are described below.

Referring back to FIG. 1, FIG. 1 illustrates a network environment 100 in which the techniques described may be applied. The network environment 100 has a network 102 that connects S servers 104-1 through 104-S, and C clients 108-1 through 108-C. As shown, several computer systems in the form of S servers 104-1 through 104-S and C clients 108-1 through 108-C are connected to each other via a network 102, which may be, for example, a corporate based network. Note that alternatively the network 102 might be or include one or more of: the Internet, a Local Area Network (LAN), Wide Area Network (WAN), satellite link, fiber network, cable network, or a combination of these and/or others. The servers may represent, for example, disk storage systems alone or storage and computing resources. Likewise, the clients may have computing, storage, and viewing capabilities. The method and apparatus described herein may be applied to essentially any type of visual communicating means or device whether local or remote, such as a LAN, a WAN, a system bus, etc. Thus, the invention may find application at both the S servers 104-1 through 104-S, and C clients 108-1 through 108-C.

Referring back to FIG. 2, FIG. 2 illustrates a computer system 200 in block diagram form, which may be representative of any of the clients and/or servers shown in FIG. 1. The block diagram is a high level conceptual representation and may be implemented in a variety of ways and by various architectures. Bus system 202 interconnects a Central Processing Unit (CPU) 204, Read Only Memory (ROM) 206, Random Access Memory (RAM) 208, storage 210, display 220, audio 222, keyboard 224, pointer 226, miscellaneous input/output (I/O) devices 228, link 229, communications 230, and port 232. The bus system 202 may be for example, one or more of such buses as a system bus, Peripheral Component Interconnect (PCI), Advanced Graphics Port (AGP), Small Computer System Interface (SCSI), Institute of Electrical and Electronics Engineers (IEEE) standard number 1394 (FireWire), Universal Serial Bus (USB), etc. The CPU 204 may be a single, multiple, or even a distributed computing resource. Storage 210, may be Compact Disc (CD), Digital Versatile Disk (DVD), hard disks (HD), optical disks, tape, flash, memory sticks, video recorders, etc. Display 220 might be, for example, an embodiment of the present invention. Note that depending upon the actual implementation of a computer system, the computer system may include some, all, more, or a rearrangement of components in the block diagram. For example, a thin client might consist of a wireless hand held device that lacks, for example, a traditional keyboard. Thus, many variations on the system of FIG. 2 are possible.

For purposes of discussing and understanding the invention, it is to be understood that various terms are used by those knowledgeable in the art to describe techniques and approaches. Furthermore, in the description, for purposes of explanation, numerous specific details are set forth in order to provide a thorough understanding of the present invention. It will be evident, however, to one of ordinary skill in the art that the present invention may be practiced without these specific details. In some instances, well-known structures and devices are shown in block diagram form, rather than in detail, in order to avoid obscuring the present invention. These embodiments are described in sufficient detail to enable those of ordinary skill in the art to practice the invention, and it is to be understood that other embodiments may be utilized and that logical, mechanical, electrical, and other changes may be made without departing from the scope of the present invention.

Some portions of the description may be presented in terms of algorithms and symbolic representations of operations on, for example, data bits within a computer memory. These algorithmic descriptions and representations are the means used by those of ordinary skill in the data processing arts to most effectively convey the substance of their work to others of ordinary skill in the art. An algorithm is here, and generally, conceived to be a self-consistent sequence of acts leading to a desired result. The acts are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the discussion, it is appreciated that throughout the description, discussions utilizing terms such as “processing” or “computing” or “calculating” or “determining” or “displaying” or the like, can refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

An apparatus for performing the operations herein can implement the present invention. This apparatus may be specially constructed for the required purposes, or it may comprise a general-purpose computer, selectively activated or reconfigured by a computer program stored in the computer. Such a computer program may be stored in a computer readable storage medium, such as, but not limited to, any type of disk including floppy disks, hard disks, optical disks, compact disk-read only memories (CD-ROMs), and magnetic-optical disks, read-only memories (ROMs), random access memories (RAMs), electrically programmable read-only memories (EPROM)s, electrically erasable programmable read-only memories (EEPROMs), FLASH memories, magnetic or optical cards, etc., or any type of media suitable for storing electronic instructions either local to the computer or remote to the computer.

The algorithms and displays presented herein are not inherently related to any particular computer or other apparatus. Various general-purpose systems may be used with programs in accordance with the teachings herein, or it may prove convenient to construct more specialized apparatus to perform the required method. For example, any of the methods according to the present invention can be implemented in hard-wired circuitry, by programming a general-purpose processor, or by any combination of hardware and software. One of ordinary skill in the art will immediately appreciate that the invention can be practiced with computer system configurations other than those described, including hand-held devices, multiprocessor systems, microprocessor-based or programmable consumer electronics, digital signal processing (DSP) devices, set top boxes, network PCs, minicomputers, mainframe computers, and the like. The invention can also be practiced in distributed computing environments where tasks are performed by remote processing devices that are linked through a communications network.

The methods of the invention may be implemented using computer software. If written in a programming language conforming to a recognized standard, sequences of instructions designed to implement the methods can be compiled for execution on a variety of hardware platforms and for interface to a variety of operating systems. In addition, the present invention is not described with reference to any particular programming language. It will be appreciated that a variety of programming languages may be used to implement the teachings of the invention as described herein. Furthermore, it is common in the art to speak of software, in one form or another (e.g., program, procedure, application, driver, . . . ), as taking an action or causing a result. Such expressions are merely a shorthand way of saying that execution of the software by a computer causes the processor of the computer to perform an action or produce a result.

It is to be understood that various terms and techniques are used by those knowledgeable in the art to describe communications, protocols, applications, implementations, mechanisms, etc. One such technique is the description of an implementation of a technique in terms of an algorithm or mathematical expression. That is, while the technique may be, for example, implemented as executing code on a computer, the expression of that technique may be more aptly and succinctly conveyed and communicated as a formula, algorithm, or mathematical expression. Thus, one of ordinary skill in the art would recognize a block denoting A+B=C as an additive function whose implementation in hardware and/or software would take two inputs (A and B) and produce a summation output (C). Thus, the use of formula, algorithm, or mathematical expression as descriptions is to be understood as having a physical embodiment in at least hardware and/or software (such as a computer system in which the techniques of the present invention may be practiced as well as implemented as an embodiment).

A machine-readable medium is understood to include any non-transitory mechanism for storing or transmitting information in a form readable by a machine (e.g., a computer). For example, a machine-readable medium includes read only memory (ROM); random access memory (RAM); magnetic disk storage media; optical storage media; flash memory devices; mechanical, electrical, optical, acoustical or other forms of non-transitory signals.

As used in this description, “one embodiment” or “an embodiment” or similar phrases means that the feature(s) being described are included in at least one embodiment of the invention. References to “one embodiment” in this description do not necessarily refer to the same embodiment; however, neither are such embodiments mutually exclusive. Nor does “one embodiment” imply that there is but a single embodiment of the invention. For example, a feature, structure, act, etc. described in “one embodiment” may also be included in other embodiments. Thus, the invention may include a variety of combinations and/or integrations of the embodiments described herein.

As used in this description, “substantially” or “substantially equal” or similar phrases are used to indicate that the items are very close or similar. Since two physical entities can never be exactly equal, a phrase such as ““substantially equal” is used to indicate that they are for all practical purposes equal.

It is to be understood that in any one or more embodiments of the invention where alternative approaches or techniques are discussed that any and all such combinations as might be possible are hereby disclosed. For example, if there are five techniques discussed that are all possible, then denoting each technique as follows: A, B, C, D, E, each technique may be either present or not present with every other technique, thus yielding 2̂5 or 32 combinations, in binary order ranging from not A and not B and not C and not D and not E to A and B and C and D and E. Applicant(s) hereby claims all such possible combinations. Applicant(s) hereby submit that the foregoing combinations comply with applicable EP (European Patent) standards. No preference is given any combination.

Thus a Multi-Modal Medical Imaging and Type Detection have been described.

What is claimed is:

1. A method for imaging comprising:
generating geometric information using a non-broadband radio imaging system;
sending said generated geometric information to a broadband radio imaging system;
using said broadband radio imaging system to generate dielectric constant information based on said generated geometric information.

2. An apparatus comprising:
a non-broadband radio imaging system having a geometry output; and
a broadband radio imaging system having a geometry input and a dielectric constant output, wherein said geometry input is coupled to said geometry output.

3. A method for combining a broadband radio imaging system and one or more other modalities of image processing to produce tissue classification.
